(19) United States
(12) Patent Application Publication
(10) Pub. No.: US 2006/0171992 A1
(43) Pub. Date: Aug. 3, 2006

(54) Blood glucose regulating composition

(76) Inventors: Cinderella Christina Gerhardt, Vlaardingen (NL); Preyesh Parmar, Bedfordshire (GB); Paul Thomas Quinlan, Bedfordshire (GB); Maria Catherine Tasker, Vlaardingen (NL)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/539,434

(22) PCT Filed: Oct. 29, 2003
(86) PCT No.: PCT/EP03/12030

§ 371 Date: Jan. 13, 2006

(30) Foreign Application Priority Data
Dec. 20, 2002 (EP) 02258932.9

(51) Int. Cl. A61K 38/17; A61K 47/00

(52) U.S. Cl. 424/439; 514/2

(57) ABSTRACT

The invention provides the use of a whey protein hydrolysate in an edible composition the whey protein hydrolysate being able to induce the cellular release of glucagon-like-peptides and cholecystokinins and/or increasing glucose uptake in target tissues, wherein the whey protein hydrolysate regulates blood glucose levels or results in, or is used for, improving or preventing decline in mental performance and/or for providing a sustained feeling of energy and/or for maintaining or providing a feeling of well-being during the post-prandial period in a subject consuming the composition.

### FIELD OF THE INVENTION

The present invention relates to the use of certain whey protein hydrolysates in the preparation of an edible composition for the regulation of blood glucose levels in humans or animals, in particular to provide for sustained energy levels or release.

### BACKGROUND OF THE INVENTION

The regulation of blood glucose levels is important for people who suffer from diabetes as well as for those who do not.

It is well known that the levels of glucose in the blood change with the time elapsed after food has been eaten, and, that these changes in blood glucose levels have marked effect upon the way that a subject feels. When blood glucose is elevated relative to normal fasting levels, the subject may feel more energetic and vitalised. However when the blood glucose levels fall below fasting level, the subject is more likely to feel irritable and fatigued, and will generally be less energetic and/or mentally alert and will generally be less productive. This drop in blood glucose levels is referred to as being hypoglycaemic.

It is therefore, beneficial for the subject if the blood glucose levels can be kept relatively constant over time, or at least, not be subject to sudden and significant changes. This is also referred to in the art as maintaining glycemic control. In a normal subject eating a healthy diet insulin accurately regulates blood glucose levels. However, a sedentary lifestyle, increased body weight and/or diet factors may lead to disturbed glycaemic control. Diets high in carbohydrates may cause rapid and high glucose peaks.

The glycaemic index (GI) is one physiologic basis for classifying carbohydrate-containing foods with the same amount of available carbohydrates. The glycaemic index is defined as the incremental area under the blood glucose response curve of a 50 g carbohydrate portion of a test food expressed as a percent of the response to the same amount of carbohydrate from a standard food taken by the same subject (Definition given by the FAO/WHO Expert Consultation, 1997).

The higher the value on the glycaemic index, the less ‘healthy’ in terms of controlling blood glucose levels the carbohydrate is currently, generally, considered to be. Many foods have a high glycaemic index value and so will cause a rapid, and generally significant, appearance of glucose in the blood. The glycaemic value of foods is determined by the type and amount of carbohydrate and generally increased by processing or refining.

It is known, for example from “The development of glucagon-like-peptide-1 pharmaceuticals as therapeutic agents for the treatment of diabetes” by D. Drucker, published in Current Pharmaceutical Design, 2001, 7, 1399-1412 and from “Determinants of the effectiveness of glucagon-like-peptide-1 in type 2 diabetes” by Toft-Nielsen et al, published J Clin Endocrinol Metab, 2001, Aug, 86(8):3853 that GLP-1 is released from gut endocrine cells following nutrient ingestion and that exogenous administration of GLP-1 lowers blood glucose in normal subjects and in patients with type 2 diabetes.

In the article “Effect of 6-week course of glucagon-like-peptide-1 on glycaemic control, insulin sensitivity and β-cell function in type 2 diabetes” by Zander et al, published in The Lancet, vol 359, Mar. 9, 2002 it is reported that GLP-1 may be given directly to patients to treat type 2 diabetes as such patients have lower levels of secretion of GLP-1 than is normal.

WO 01/37850 discloses compositions comprising a partially purified non-whey milk protein hydrolysate which is enriched in caseino-glycomacropeptide, inducing the release of glucagon-like-peptide 1 (GLP-1) which can be used to treat diabetes. It is also disclosed in WO 01/37850 that proglucagon, synthesised by L-cells found in the distal ileum and colon, is known to be post-translationally processed into peptides including glucagon-like peptide-I (GLP-1), a potent insulin secretagogue. In addition to potentiating glucose-induced insulin secretion, GLP-I is known to stimulate proinsulin gene expression and proinsulin biosynthesis.

Other actions of GLP-1 include inhibition of glucagon secretion and gastric motility (emptying). GLP-1 can bind to GLP-1R receptors in the brain, promoting satiety and suppressing food intake. Increasing insulin sensitivity is a key goal in the treatment of Type 2 diabetes and stimulation of endogenous release of GLP-1 is a potential alternative to intravenous administration.

U.S. Pat. No. 6,207,638 and US 2002/0019334 disclose nutritional compositions stimulating the release of CCK. The composition comprise a) a protein selected from casein, whey and soy, b) a glycomacropeptide, c) a long chain fatty acid, and d) soluble and insoluble fibers. Whey protein hydrolysates are not disclosed. The compositions may be used to help people with type II diabetes maintain glycemic control and extend satiety. In US 2001/0021694, from the same inventor there are disclosed compositions which are used to help people with type 2 diabetes maintain glycemic control, the compositions comprising casein (glyco)macropeptide or a hydrolysis product thereof.

WO 02/15719 discloses nutritional compositions comprising whey proteins which may be at least in part hydrolysed. The inclusion of the whey protein hydrolysates is stated to result in reduced satiety effects from the compositions. The nutritional compositions are intended for people suffering from reduced appetite such as those convalescing and anorexia suffers. There is no disclosure of the control of blood glucose or of the treatment of individuals suffering from diabetes.

WO 01/85984 (Davisco Foods International, Inc) discloses whey protein hydrolysates having an increased ACE—suppressing activity in mammals. There is no disclosure of the control of blood glucose levels or of the treatment of individuals suffering from diabetes.

US 2002/0037830 discloses the use of a whey protein hydrolysate in the preparation of an additive for use as an energy supplement or metabolic nutrient.

Aoyama et al in the paper “Effect of soy and milk whey protein isolates and their hydrolysates on weight reduction in genetically obese mice”, Biosci, Biotechnol, Biochem., 64(12), 2594-2600, 2000 discuss the effect on genetically obese mice of a milk whey protein isolate and its hydrolysates. The isolates and hydrolysates were found to be less effective than soy proteins.

JP 04 149139 discloses hypoglycaemic agents obtained by the enzymic hydrolysis of milk protein for treating diabetes where blood sugar level is controlled.

EP-A-629 350 discloses the use of cow's milk protein hydrolysates which are substantially free of allergenic proteins for the prophylaxis or treatment of type 1 diabetes mellitus in children.

Powders to produce drinks comprising β-lactoglobulin and α-lactalbumin, and drinks produced therefrom, are known for blood pressure lowering applications. A powder produced by Davisco Foods International (Minnesota, USA) comprises 20 g of β-lactoglobulin and α-lactalbumin, 1 g of fat and 6 g of carbohydrate per 30 g of powdered product. The powders can be mixed with water or milk to produce the drink. No disclosure is made of use in blood glucose control or diabetes applications. The powders and drinks provide over 55% of the total calories in the powder or drink (when made with water or cows milk) from the protein content.

Whey based energy drinks are also known in the art. Designer Whey Protein Blast drinks (ex Next Proteins, California, USA) comprise β-lactoglobulin and α-lactalbumin and are used as food supplements for building muscle mass. The drinks comprise very low levels of carbohydrates and no fat and thus the calories are provided predominantly from the protein. A bottle of 20 American ounces (about 600 ml) of the drink comprises no fat, 1 g carbohydrate and 17 g protein.

However, despite the above developments, there is still a need in the art for edible (nutritional or therapeutic) compositions which may be administered orally, preferably as a food composition, and which can be used for the regulation of blood glucose levels in humans or animals. In particular there is a need for such compositions which have improved efficacy over the known treatment compositions or which are derived from additional sources, or, which are in a more convenient form for a subject to take. Furthermore, there is a need for such compositions which can be used as part of a normal, daily, diet. In particular, there is a need for compositions that can be used as meal replacement products or snack foods.

There is also a need to provide such edible compositions that have an acceptable taste e.g. the compositions are not too sweet or too bitter and can easily be formulated into edible compositions as well as providing the above effects.

The present invention seeks to address one or more of the above-mentioned problems.

Recognising the demand for efficient and convenient products to be used in the regulation of blood glucose levels, research has been carried out by the inventors to find compounds that are effective in these applications and which can be used in edible compositions, especially food compositions of the type eaten in a typical diet.

In particular, it is an object of the invention to provide edible compositions that can be used in the regulation of blood glucose levels to provide beneficial effects in the feeling of energy, well being or mood.

It is also an object of the invention to provide edible compositions that exhibit greater efficacy in the regulation of blood glucose levels then conventional edible compositions.

It is also an object of the invention to provide such compositions which are in a convenient form for consumers and which have acceptable taste and which can be consumed as part of a normal daily diet and which are not only available in ‘medicament’ form.

### SUMMARY OF THE INVENTION

Surprisingly, it has now been found that whey protein hydrolysates (WPH) that stimulate the cellular relase of GLP-1 and CCK and/or increase glucose uptake in target issues are especially suitable for use in the regulation of blood glucose levels.

Without wishing to be bound by theory, it is believed that because these WPH stimulate the cellular release of more than one peptide, one of which is involved in controlling the levels of glucose (GLP-1) in the blood and the other which is involved in digestion (CCK) they are particularly effective. Moreover both GLP-1 and CCK slow down gastric emptying directly leading to a ‘slowing-down’ of glucose absorption into the blood.

Furthermore, there is a direct stimulatory effect of the WPH on glucose uptake in target tissues such as muscles, liver and fat cells, possibly by increasing insulin sensitivity. In particular it has been found that better glycaemic control is achieved which results in reduced peak hyperglycaemic response and/or in reduced variability in glucose response and/or in prolonged post-prandial glucose. In other words, the glycaemic response is extended.

It has also been found that the WPH of the invention exhibit an increased level of induced cellular GLP, especially GLP-1, release at a given concentration than do other milk proteins, milk protein hydrolysates or non-hydrolysed whey proteins.

According to a first aspect, the present invention provides the use of a whey protein hydrolysate in an edible composition the whey protein hydrolysate being able to induce the cellular release of glucagon-like-peptides and cholecystokinins and/or increasing glucose uptake in target tissues, wherein the whey protein hydrolysate regulates blood glucose levels or results in, or is used for, improving or preventing decline in mental performance and/or for providing a sustained feeling of energy and/or for maintaining or providing a feeling of well-being during the post-prandial period in a subject consuming the composition.

According to a second aspect, the present invention provides a method of regulating blood glucose levels, improving or preventing decline in mental performance, providing a sustained feeling of energy or maintaining or providing a feeling of well-being during the post-prandial period, which method comprises the step of orally administering to a subject by means of an edible composition an effective amount of a whey protein hydrolysate which is capable of inducing the cellular release of glucagon-like-peptides and cholecystokinins and/or increasing glucose uptake in target tissues.

By “improving or preventing a decline in mental performance” as referred to herein is meant that a subject exhibits or experiences an actual or perceived positive effect on performance in mental tasks in the post-prandial period after consuming a composition comprising the claimed whey protein hydrolysates.

By “a sustained feeling of energy” as referred to herein is meant that a subject exhibits or experiences an actual or perceived effect of feeling energetic in the post-prandial period after consuming a composition comprising the claimed whey protein hydrolysates.

By a “feeling of wellbeing” as used herein is meant that a subject exhibits or experiences an actual or perceived feeling of being in a good mood in the post-prandial period after consuming a composition comprising the claimed whey protein hydrolysates.

A “flowable” product as referred to herein is a liquid, semi-liquid, powdered or particulate product which when poured with or without the application of pressure flows out of a container even if the product does not flow out in a continuous stream as may occur with semi-liquid, powdered or particulate products. The term does not include products which are in one piece as these are not capable of flowing out of a container, nor, products which are eaten in a physical state which does not flow such as ice-cream.

The liquid or flowable edible compositions of the invention are effective in the control of blood glucose levels and have acceptable sensory properties (such as acceptable taste) and have a good balance of the level of whey protein hydrolysate used and the level of calories in the product obtained from protein.

The preferred whey protein hydrolysates according to both aspects of the invention comprise hydrolysates of β-lactoglobulin, α-lactalbumin or a mixture thereof.

The use of the WPH which induce the cellular release of both CCK and GLP and/or increase glucose uptake in target tissues, in the preparation of edible compositions to be used in the regulation of blood glucose levels has the advantages that it provides compositions which are effective for these purposes, which can be administered orally and which have acceptable taste, and which can conveniently be used as a part of a daily diet. Moreover, for the WPH which induce the cellular release of both CCK and GLP, the effect is advantageous when compared to the effect obtained from the consumption of a product that comprises WPH which only induce the release of either CCK or GLP. It is believed that the combined release (either simultaneously or stepwise) of these two peptides results in an more effective control of blood glucose levels. Furthermore this is believed to result in a direct stimulatory effect of upon glucose uptake in target tissues such as muscles, liver and fat cells.

Without wishing to be bound by theory, it is believed that the good regulation of blood glucose levels achieved by the invention occurs because of one or more of the following:

the whey protein hydrolysates are capable of inducing the cellular release of both glucagon-like-peptides and cholecystokinins. This is believed to result in slower gastric emptying which in turn slows down the absorption of glucose into the blood stream which results in better glycaemic control as the blood glucose level is more constant over time, or

the WPH above, because of the stimulation of GLP release, stimulate insulin secretion from pancreatic β-cells resulting in better glycaemic control, or.

the WPH which are capable of increasing glucose uptake in target tissues lead to better glycaemic control.

The above is especially beneficial for those who need to control blood glucose levels e.g. those suffering from Type 2 diabetes. It also helps to prevent the deterioration of people who have glucose intolerance and so lessen the chances of them developing Type 2 diabetes. Furthermore, this has also been found to provide other advantages including; improved mental performance and/or a sustained feeling of energy and/or being less likely to feel irritable, in the post-prandial period.

“The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.”

Except in the operating and comparative examples, or where otherwise explicitly indicated, all numbers in this description indicating amounts of material or conditions of reaction, physical properties of materials and/or use are to be understood as modified by the word “about”. All amounts are as percentages by weight unless otherwise stated. For the edible compositions, all percentages are by weight based on the total weight of the composition unless otherwise stated.

### DETAILED DESCRIPTION

Peptide Secretion by the WPH

Cholecystokinin or “CCK” as referred to herein include all peptides of the CCK family, including CCK-4, CCK-8, CCK-22, CCK-23, CCK-24, CCH-25, CCK-36, CCK-27, CCK-28, CCK-29, CCK-30, CCK-31, CCK-32, CCK-33, CCK-39, CCK-58.

Glucagon-like-peptides (GLP) and “GLP” as used herein include all peptides of the GLP family including those of GLP-1 and GLP-2. GLP-1 has been found to be especially of interest because of its effect on insulin secretion.

Cellular Release

Inducing the cellular release of the peptides as described herein refers to inducing the release thereof by suitable cells, preferably gastrointestinal cells, after the interaction of the whey protein hydrolysate (WPH) with those cells.

Inducing the cellular release of the peptides according to the invention can be measured in vitro, for example by the use of an intestinal cell line. Suitable cell lines are well known in the art. The cells used in the examples are GLUTag cells which are an L cell line from intestinal endocrine tumors arising in the large bowel in proglucagon-simian virus 40 large T antigen transgenic mice. These cells are commercially available and are further described in the publication by Drucker D. J. et al (1994): Activation of proglucagon gene transcription by protein kinase A in a novel mouse enteroendocrine cell line, Mol Endocrinol 8:1646-1655.

Examples 1 and 2 further illustrate the in vitro cellular release of CCK and GLP-1. The information in these examples is incorporated by reference in this section.

When a subject (animal or human) ingests the claimed WPH, either by itself or as part of an edible composition, the cellular release of CCK and GLP in the body is stimulated resulting in the effects according to the invention.

This cellular release can also be measured in vivo, for example, by measuring the increase or appearance of CCK and GLP levels in the blood of that subject after consumption of the WPH or an edible composition comprising it. Suitable techniques for measuring the CCK and GLP levels in the blood are well known in the art and do not need to be further described here.

The WPH of the invention show cellular release of CCK and GLP-1 in the in vitro cellular release test of examples 1 and 2 particularly when used at a concentration of at least 5 mg/ml.

Glucose Uptake in 3T3L1 Adipocytes

Stimulating glucose uptake into adipocytes as described herein refers to stimulating glucose uptake into suitable cells, preferably insulin sensitive target cells like adipocytes, muscle cells and liver cells after the interaction of the whey protein hydrolysate with those cells.

Stimulating the cellular uptake of glucose according to the invention can be measured in vitro, for example by the use of adipocytes. Suitable cell lines are well known in the art. The cells used in the examples are 3T3L1 cells that have been differentiated into adipocytes in vitro. These cells are commercially available from the American Tissue Culture Collection.

Examples 3 and 4 further illustrate the in vitro cellular uptake of [³H] glucose. The information in these examples is incorporated by reference in this section.

When a subject (animal or human) ingests the claimed WPH, either by itself of as part of an edible composition, the cellular uptake of blood glucose by target cells is stimulated according to the invention.

The WPH of the invention shows stimulation of glucose uptake as in the in vitro cellular uptake test of example 3 at a concentration of at least 100 μg/ml. In the presence of insulin as in example 3 the potency of WPH to stimulate glucose uptake increases to at least 10 μg/ml, suggesting that WPH enhances the sensitivity of the cells for insulin.

The Whey Protein Hydrolysate

The terms “whey protein hydrolysate which is capable of inducing the cellular release of glucagon-like-peptides and cholecystokinins”, and “WPH” as used herein include all of the following; a single whey protein hydrolysate which induces the cellular release of both the aforementioned peptides, a mixture thereof, a mixture of two or more whey proteins hydrolysates wherein the mixture induces the cellular release of both peptides even if at least one of the components induces the cellular release of only one of the peptides. The same comments apply for the increasing glucose uptake in target tissues References herein to WPH are used to refer to both the singular and the plural use of whey protein hydrolysate as described above.

The WPH may comprise any whey protein which has been hydrolysed and which is capable of inducing the cellular release of glucagon-like-peptides and cholecystokinins and/or increasing glucose uptake in target tissues.

Suitable methods of hydrolysis of the whey protein include chemical methods (for example by acid hydrolysation) or enzymatical methods (including peptidases and bacterial or plant proteases) or by treatment with bacterial cultures. Examples of suitable enzymes which can be used to hydrolyse a whey protein include pepsin, trypsin and chymotrypsin.

It is especially preferred that the WPH comprises hydrolysates of β-lactoglobulin or α-lactalbumin, most preferably mixtures thereof. The weight ratio of these hydrolysates in the mixture is preferably in the range of from 5:1 to 1:5, more preferably 4:1 to 1:4, such as 3.5:1 to 1:2.

One particular WPH which may be used according to the invention comprises from 5 to 20% by weight of aspartic acid, 10 to 25% by weight of leucine, 5 to 20% by weight of lysine and 10 to 32% by weight of glutamic acids.

The WPH may have a degree of hydrolysis in the range of up to 20%, preferably of from 1 to 15% or 20%, more preferably of from 2 to 10%, such as 5 to 9%. The degree of hydrolysis is determined by OPA methodology (Lee K S, Drescher D G., Fluorometric amino-acid analysis with o-phthaldialdehyde (OPA), Int. J. Biochem. 1978; 9(7): 457-467).

The WPH preferably has a weight average molecular weight in the range of from about 1000 Dalton to 12000 Dalton, preferably of from 2000 Dalton to 8000 Dalton. It is preferred that 4 to 40% by weight, more preferably 10 to 30% of the WPH has a weight average molecular weight in the rage of from 2000 to 5000 Daltons and/or 1 to 30% by weight, more preferably 2 to 20% of the WPH has a weight average molecular weight in the range of from 5000 to 10000 Daltons.

The WPH preferably have a pH in the range of from 6 to 9 at 20° C. in a 10 mg/ml solution in de-ionised water, more preferably of from 6.5 to 8.

The WPH which may be used according to the invention are known in the art and are commercially available. A description for one method to obtain suitable WPH is described in WO 01/85984 A1. A suitable commercially available source of the WPH is the Biozate™ whey protein hydrolysate products from Davisco Foods Inc, Minnesota, USA. The product designated “Biozate™ 1” has been found to be especially suitable.

The WPH is used in the preparation of edible compositions. The term “preparation” as used herein includes all suitable techniques of producing edible compositions, for example, mixing, blending, homogenising, high-pressure homogenising, emulsifying, dispersing, or encapsulating. The WPH may be included in the edible composition by any suitable method known in the art and these methods will depend upon the type of edible composition.

The WPH may be micro-filtered or ion-exchanged (either as the hydrolysate or as the parent protein). It may be enhanced with glutamine, alanine, cystine and branched chain amino acids.

Method of Administering the WPH

The invention also provides a method for the regulation of blood glucose levels by orally administering an effective amount of the WPH.

The total effective amount of WPH administered according to the method may vary according to the needs of the person to whom it is administered. Typically total amounts of from 0.1 g to 150 g will be administered, preferably 1 g to 80 g, more preferably 5 g to 50 g. The effective daily amount may be administered by a single dose or by multiple doses daily.

The WPH may be administered to a human or animal subject in any suitable form, for example as a capsule, tablet, solution, or, preferably as an edible food composition as described herein including bar products, beverage products and liquid products such as ready-to-drink products.

The Edible Composition

The edible composition may be in the form of a nutritional supplement (such as a tablet, powder, capsule or liquid product), a food composition (product), a beverage, or a meal replacement product.

A nutritional supplement as used herein refers to a composition or supplement which provides at least one beneficial agent such as vitamins, minerals, trace elements, the WPH etc and which is intended to supplement the amount of such agents obtained through normal dietary intake. These compositions or supplements do not generally contain a significant amount of calories, protein, carbohydrate or fat. They are not intended to be taken as a food but rather as a supplement to the daily diet.

A food composition according to the invention may be any food which can be formulated to comprise the WPH. Preferably it contains a total of at least 5% by weight of at least one of protein, fat, and carbohydrate or a mixture thereof or has a calorie content of at least 10 kilocalories per serving or 100 g, preferably of at least 20 kilocalories. A food composition does not encompass nutritional supplements as described above.

Food compositions according to any aspect of the invention may suitably be selected from dairy based products (such as milk based products and drinks), soy based products, breads and cereal based products (including pasta and cereal bars), cakes, biscuits, spreads, oil-in-water emulsions (such as dressings, ketchup and mayonnaise), ice creams, desserts, soups, powdered soup concentrates, sauces, powdered sauce concentrates, beverages, sport drinks, health bars, fruit juices, confectionery, snack foods, ready-to-eat meal products, pre-packed meal products, and dried meal products etc.

A meal replacement product as used herein refers to a product which is intended to replace one or more conventional meals a day; they are of a controlled calorie content and are generally eaten as a single product. However several such products may be eaten together. Examples of meal replacement products and products to be used as part of a meal replacement plan include; (ready-to-drink) liquid products such as milk or soya-based drinks, soluble powders used to prepare those drinks and drinks prepared therefrom, bars, soups, cereal or noodle or pasta-based products, desserts such as rice puddings, custards and the like and porridge and the like. Meal replacement products are generally used by consumers following a calorie controlled diet or wishing to control their body weight.

Meal replacement products and products to be used as a part of a meal replacement plan are especially preferred according to the invention. They have been found to be especially suitable as they can provide good satiety effects combined with restricted calorie content in a convenient form. It is especially preferred that the meal replacement product is a ready-to-drink liquids, a soluble powder used to prepare drinks, a liquid produced therefrom, a soup, a dessert, a bar, a cereal based or pasta based or noodle based product, or, a soluble powdered product.

The edible composition may be for example; a solid product, a powdered product, a tablet, a capsule, a liquid, a flowable, spoonable, pourable or spreadable product or a bar etc. The edible composition may be a powder which is mixed with a liquid, such as water or milk, to produce a liquid or slurry product such as a meal replacement product, or a product to be used as part of a meal replacement plan.

The edible compositions preferably comprise a total amount of from 0.1% to 80% by weight of the WPH based on the weight of the composition, preferably 0.5 to 40% wt, more preferably 1 to 30% wt, most preferably 2 or 5 to 20% wt. The edible compositions preferably comprise an amount of from 0.1 to 80%, preferably 1 to 50%, by weight of hydrolysates of β-lactoglobulin, α-lactalbumin or mixtures thereof based on the weight of the composition.

According to one embodiment of the invention, the edible compositions may comprise less than 20 g in total per serving, or per product where the product is used as a single serving, of the WPH whether or not the above-mentioned amounts are used.

If the edible composition is a liquid or readily flowable composition, such as liquid meal replacement product or a soup, then the total amount of WPH will preferably be in the range of from 0.1 to 40 or 50% by weight, more preferably 1 to 40% wt, most preferably 2 to 30% wt based on the total weight of the composition. It is preferred that these compositions comprise a total amount of from 0.1 to 40% by weight based on the weight of the composition of the WPH and 40% or less of the total calories in the edible composition are provided by the WPH.

If the edible composition is a solid composition, such as a bar product, e.g. a bar meal replacement product, the amount of WPH will typically be in the range of from 0.1 to 80% by weight, preferably 0.5 to 40% by weight based on the total weight of the composition. It is especially preferred that the bar compositions comprise hydrolysates of β-lactoglobulin, α-lactalbumin or a mixture thereof in a total amount of from 0.1 to 80% wt, more preferably 1 to 10% wt, based on the weight of the composition.

The edible composition will typically comprise proteins, preferably in an amount of from 0.1 to 30 or 40% by weight of the edible composition. It is preferred that the compositions comprise 0.5 to 25% wt of protein, preferably 1 to 20% wt. In the liquid or flowable compositions the protein present provides up to 50% of the total calories of the edible composition, more preferably between 20% and 50%, most preferably between 25% and 50%. For the other types of edible compositions, these amounts are preferred but are not essential.

The edible composition may comprise fats, preferably in an amount of up to 60 or 70% by weight based on the weight of the composition, more preferably from 0.5 to 30 or 35% wt, most preferably from 2 to 20% fat. Any suitable fat may be used for example, vegetable fats, plant oils, nut oils, seed oils, or mixtures thereof. Saturated or unsaturated (mono-unsaturated and poly-unsaturated) fats may be used.

The edible compositions may also comprise one or more carbohydrates, preferably in an amount of from 1 to 95% by weight based on the weight of the composition, more preferably 5 to 70% wt, most preferably 10 to 60% wt, such as 15 to 50% wt. Any suitable carbohydrate may be used, for example sucrose, lactose, glucose, fructose, corn syrup, maltodextrins, starch, modified starch or mixtures thereof.

The edible composition may also comprise dietary fibres, for example in an amount of from 0.1 to 40 or 50% by weight based on the weight of the composition, preferably 0.5 to 20% wt.

The edible composition may comprise dairy products such as milk, yoghurt, kefir, cheese or cream for example in an amount up to 70% by weight based on the weight of the composition, preferably 1 to 50% wt. Alternatively the edible composition may be soy-protein based used in the same amounts. The inclusion of these ingredients will be chosen so that the desired amount of protein, fat and carbohydrates etc are included in the edible composition;

The edible composition may comprise one or more emulsifiers. Any suitable emulsifier may be used, for example lecithins, egg yolk, egg-derived emulsifiers, diacetyl tartaric esters of mono, di or tri-glycerides or mono, di, or triglycerides. The composition may comprise of from 0.05 to 10% by weight, preferably from 0.5% to 5% wt of the emulsifier based on the weight of the composition.

The edible composition may also comprise stabilisers. Any suitable stabiliser may be used, for example starches, modified starches, gums, pectins or gelatins. The composition may comprise of from 0.01 to 10% by weight, preferably 1 to 5% wt of stabiliser based on the weight of the composition.

The edible composition may comprise up to 60% by weight of fruit or vegetables particles, concentrates, juice or puree based on the weight of the edible composition. Preferably the compositions comprise 0.1 to 40% wt, more preferably 1 to 20% wt of these ingredients. The amount of these ingredients will depend upon the type of edible composition; for example soups will typically comprise higher levels of vegetables than will a milk based meal replacement drink.

The edible composition may also comprise 0.1 to 30% by weight of salts based on the weight of the composition, preferably 0.5 to 15% wt, more preferably from 3 to 8% wt. Any edible salts may be used, for example, sodium chloride, potassium chloride, alkali metal or alkaline earth metal salts of citric acid, lactic acid, benzoic acid, ascorbic acid, or, mixtures thereof.

The edible composition may comprise one or more cholesterol lowering agents in conventional amounts. Any suitable, known, cholesterol lowering agent may be used, for example isoflavones, phytosterols, soy bean extracts, fish oil extracts, tea leaf extracts.

The edible composition may comprise up to 10 or 20% by weight, based on the weight of the composition, of minor ingredients selected from added vitamins, added minerals, herbs, spices, flavourings, aromas, antioxidants, colourants, preservatives or mixtures thereof. Preferably the compositions comprise of from 0.5 to 15% by weight, more preferably 2 to 10% of these ingredients. It is especially preferred that the compositions comprise added vitamins and minerals. These may be added by the use of vitamin premixes, mineral premixes and mixtures thereof. Alternatively the vitamins and/or minerals may be added individually. These added vitamins and/or minerals are preferably selected from at least one of vitamins A, B1, B2, B3, B5, B6, B12, C, D, E, H, K or minerals calcium, magnesium, potassium, zinc and iron.

The amounts of protein, fat, carbohydrate and other ingredients in the edible composition will vary according to the product format of the composition and also, where required, according to national or regional legislation.

If the edible composition is a meal replacement product then the calorie content of the product is preferably in the range of from 50 calories to 600 calories, more preferably 100 calories to 500 calories, most preferably 200 calories to 400 calories.

The compositions may be made by any suitable method known in the art; such methods are well known to those skilled in the art and do not need to be described further here.

The edible compositions are intended for oral consumption and may be consumed by a human or an animal in connection with any one or more of the following; to regulate blood glucose levels including for maintaining or improving mental performance, and/or for providing a sustained feeling of energy and/or for maintaining or providing a feeling of well-being during the post-prandial period in a subject consuming the composition.

A nutrient as referred to herein may be any component of a food product from which the consumer derives physiological benefit. Examples include macro-nutrients such as carbohydrates, fats and proteins or micro-nutrients such as vitamins, minerals, and trace elements. Fibres, although not absorbed by the body, are considered herein as nutrients. Water, although it provides a benefit to the body, is not considered as a nutrient.

The consumption of a composition comprising the WPH according to the invention may occur as part of a programme followed on medical advice or upon the desire of a consumer. The compositions are preferably used as part of a dietary plan or a weight management plan. In the latter case, the consumer may be seeking a composition which will help in the regulation of blood glucose levels and help to avoid peaks and troughs therein which generally occur during the day for most people, for example to maintain energy levels. A dietary plan. As referred to herein is a plan followed by those who are not following the plan for the purpose of controlling body weight. A weight management programme is one followed by those for the purpose of controlling body weight.

It is especially advantageous if the composition is a meal replacement composition that is intended to be used as part of a weight control plan, as glucose tolerance improves when a subject looses weight or maintains a healthy body weight.

Another advantage of the present invention is that aids in blood glucose regulation through edible food compositions rather than needing to be provided as a medication.

The invention is further described by way of the following examples which are to be understood as not limiting. Further examples within the scope of the invention will be apparent to the person skilled in the art.

### EXAMPLES

### Examples 1 and 2

### Stimulated Release of GLP 1 and CCK in Cultured GLUTag Cells

1. Materials

a) Whey Protein Hydrolysate:

The whey protein hydrolysate used was Biozate™ 1 which is a commercially available material from Davisco Foods International Inc., Le Sueur, Minesotta, U.S.A. Biozate™ 1 comprises a mixture of hydrolyzed β-lactoglobulin and α-lactalbumin.

The technical specification of Biozate™ 1 is given below. The pH is 8.0. The degree of hydrolysis, as measured by the OPA method referred to hereunder, is 5.5+/−1.5. The molecular weight profile (Daltons) is: 30 to 45% greater than 10,000, 7 to 12% in the range 5000 to 10000, 15 to 25% in the range 2000 to 5000, 30-45% less than 2000 as measured by SEC-HPLC.

b) GLUTag Cells:

The GLUTag cells were obtained under license from Toronto General Hospital, Toronto, Canada. GLUTag cells are an L cell line from intestinal endocrine tumors arising in the large bowel in proglucagon-simian virus 40 large T antigen transgenic mice. These cells are further described in the publication by Drucker D. J. et al (1994): Activation of proglucagon gene transcription by protein kinase A in a novel mouse enteroendocrine cell line. Mol Endocrinol 8:1646-1655.

C) Materials for Cell Culture:

Dulbecco's Modified Eagles Medium (DMEM) and foetal bovine serum (FBS) were obtained from Invitrogen Ltd (Paisley, Scotland, UK).

2. Method

GLUTag cells were grown during incubation at 37° C. in DMEM containing 10% (vol/vol) FBS. The medium was changed every 3 to 4 days until cell confluence was achieved. The cells were then trypsinized, plated in 24-well cultures plates (0.5×10⁵ cells/well) and the plates were stored under the same incubation conditions as described above. After 3 days storage the cells were washed twice with DMEM containing 0.5% (vol/vol) FBS and then, to four series (A to D) of 3 wells, different amounts of Biozate™ 1 were added as detailed below. Thus, each series was prepared in triplicate. A control sample which did not have any added Biozate™ 1 was also prepared in triplicate.

Series A—0.5 mg/ml Biozate™ 1

Series B—3 mg/ml Biozate™ 1

Series C—5 mg/ml Biozate™ 1

Series D—10 mg/ml Biozate™ 1

The plates were incubated as detailed above and after 1 hour incubation an aliquot was taken from each plate to measure CCK release. A further aliquot was taken from each plate after 2 hours incubation to measure GLP-1 release. The aliquots were treated as detailed below before being tested to determine CCK or GLP-1 release.

The aliquots were collected and 50 μg/ml phenylmethanesulfonyl fluoride (PMSF) was added thereto. The aliquots were frozen at −80° C. for subsequent analysis for CCK and GLP-1 secretion. The aliquots were defrosted and centrifuged (5000 g) to remove cell debris. The CCK and GLP-1 release from the GLUTag cells was then tested.

CCK release was measured using a commercial enzyme immunoassay kit (from Phoenix Pharmaceuticals, Belmont, Calif., USA) which measures CCK 26-33 non-sulfated and sulfated. According to the test kit specifications, the intra-assay variation is <5% and the inter-assay variation is <14%.

GLP-1 release was measured using a commercial ELISA kit (from Linco Research Inc., St Charles, Mo., USA). This kit measures biologically active forms of GLP-1 [i.e. GLP-1 (7-36 amide) and GLP-1 (7-37)]. Prior to measuring GLP-1 release, the aliquots were diluted 1 parts to 10 parts with DMEM containing 0.5% (vol/vol) FBS to bring the GLP-1 concentration within the standard detection range of the ELISA kit.

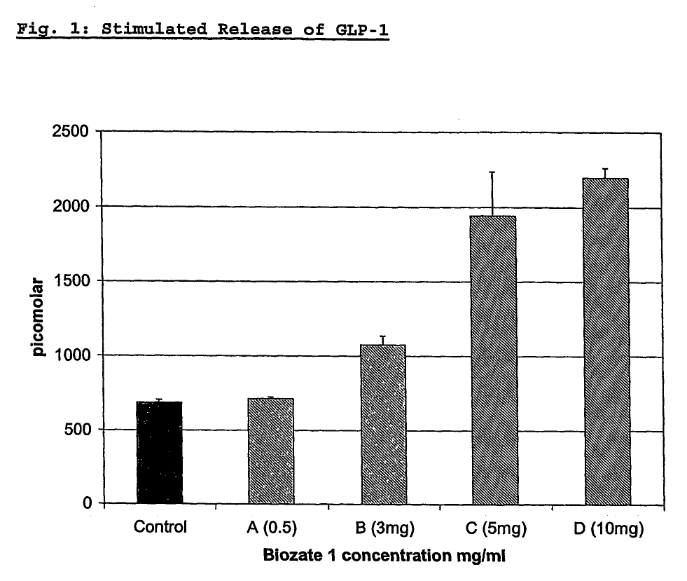

FIG. 1 shows the concentration of GLP-1 secreted from GLUTag cells into the media after 2 hours incubation at 37° C. with the Biozate™ 1.

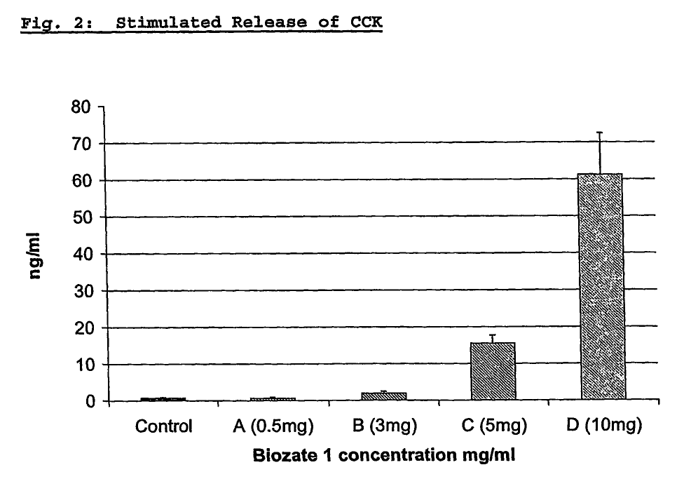

FIG. 2 shows the concentration of CCK secreted from GLUTag cells into the media after 1 hour incubation at 37° C. with Biozate™ 1.

On both FIGS. 1 and 2, the x axis shows the series and the concentration of Biozate™ 1 used. The y axes of FIGS. 1 and 2 show the concentration of GLP-1 or CCK secreted from GLUTag cells into the media after incubation. For FIG. 1 the concentration is expressed in pico moles per litre (10⁻¹² M) and for FIG. 2 in nanograms/ml.

Cell viability was positively determined using the CytoTox 96® non-radioactive cytotoxicity assay (Promega, Madison, USA) in order to prove that peptide release was not due to cell death.

From the results in FIGS. 1 and 2, it can be seen that the whey protein hydrolysate used (a mixture of β-lactoglobulin and α-lactalbumin hydrolysates) results in the release of both GLP-1 and CCK from the GLUTag cells into the media.

### Example 3

### 3H-Deoxy-glucose uptake in 3T3L1 adipocytes at 0 and 1 nM levels of insulin

1. Materials

a) Whey Protein Hydrolysate:

The whey protein hydrolysate used was Biozate™ 1 as detailed for examples 1 and 2. Biozate™ 1 was prepared by dissolving it in serum-free assay medium at a concentration of 10 mg/ml. From this 6 further dilutions were prepared, each 10 times more dilute than the previous one.

b) 3T3L1 Cells:

Mouse embryo derived 3T3L1 cells (CL-173, sourced from American Tissue Culture Collection) were used.

c) Materials for Cell Culture:

Assay medium: Dulbecco's Modified Eagles Medium (DMEM) and foetal bovine serum (FBS) were obtained from Invitrogen Ltd (Paisley, Scotland, UK). DMEM was supplemented with 10% foetal calf serum, 2 mM L-glutamine and 1% penicillin & streptomycin.

A serum-free assay medium was prepared (SFAM) by supplementing DMEM with 2 mM L-glutamine and 1% penicillin & streptomycin.

A differentiation medium (DM) was prepared by supplementing the assay medium with 250 nM dexamethasone, 5 μg/ml insulin and 0.5 mM 3-isobutyl-1-methylxanthine (IBMX).

A post-differentiation medium (PDM) was prepared by supplementing the assay medium with 5 μg/ml insulin.

Krebbs—Ringer phosphate buffer=13.6 mM NaCl, 4.7 mM KCl, 1.25 mM CaCl₂, 1.25 mM Mg₂SO₄, 10 mM Na₂HPO₄.

Phosphate Buffered Saline (PBS)

2. Methods

The mouse embryo derived 3T3L1 cells were cultured in AM routinely, with medium changes every 2-3 days. The cells were grown to 95% confluence, ensuring that the cultures did not become overfluent. At near confluence the cells were prepared for subculture into multi-well plates for experimentation or new flasks for continual passage.

For subculture, the AM was removed and discarded from the flasks. The cells are rinsed briefly with 2-3 ml of Trypsin/EDTA to remove all traces of serum. 5 ml of Trypsin/EDTA was then added to the flasks to raise the cells from the surface of the plastic. The cells were observed under an inverted microscope until the cells were dispersed (usually within 5 minutes, however the flasks were, where necessary, placed in an incubator at 37° C. for several more minutes to facilitate dispersal). Once all the cells had been raised from the flasks the tryrpsin/EDTA solution was neutralised by the addition of 5 ml of trypsin neutralising solution or AM. The cells were then transferred to centrifuge/universal tubes and centrifuged at 2500 r.p.m. for 3 minutes, the supernatant aspirated carefully, the cells re-suspended and washed in PBS at 37° C. and centrifuged once again. The PBS was aspirated carefully and the cells re-suspended in 10 ml of AM. The cells were then counted, diluted with AM and transferred to 48-well plates at concentrations of 25-30,000 cells/ml. The cells were then left untreated in the multi-well plates for 24 hours to allow the cells to adhere to the plastic.

The cells are then allowed to grow to near confluence in AM, for about 2 days. After this the medium was aspirated and replaced with DM, and maintained for a further 3 days. After three days the medium was changed to PDM for a further 2 days. At this stage the 3T3L1 cells were differentiated to adipocyte like morphology and had lipid droplets formed within the cells. These differentiated cells were then treated with the different concentrations of Biozate™ 1 for 3 days as detailed below:

Series A—100 μg/ml Biozate™ 1

Series B—10 μg/ml Biozate™ 1

Series C—1 μg/ml Biozate™ 1

Series D—100 ng/ml Biozate™ 1

Series E—10 ng/ml Biozate™ 1

Series F—1 ng/ml Biozate™ 1

Series G—100 μg/ml Biozate™ 1

After the 3 day treatment the cells were washed three times with SFAM and left in 250 μl of Krebbs buffer for 30 minutes in a incubator at 37° C. Radioactively labelled glucose (3H-deoxy glucose) was added to the cells (2.5 μCi/well) and the cells incubated for another hour. The cells were then washed three times with ice cold SFAM. The cells were then lysed with 500 μl/well of warmed 0.1% wt Triton X-100 for one hour.

100 μl of lysate from each of the wells was counted by liquid scintillation counting to assess the amount of radio-labelled glucose taken up by the adipocytes. The washes were also counted to ensure that most of the unincorporated radio-labelled glucose was removed from the multi-well plates before the adipocytes were lysed.

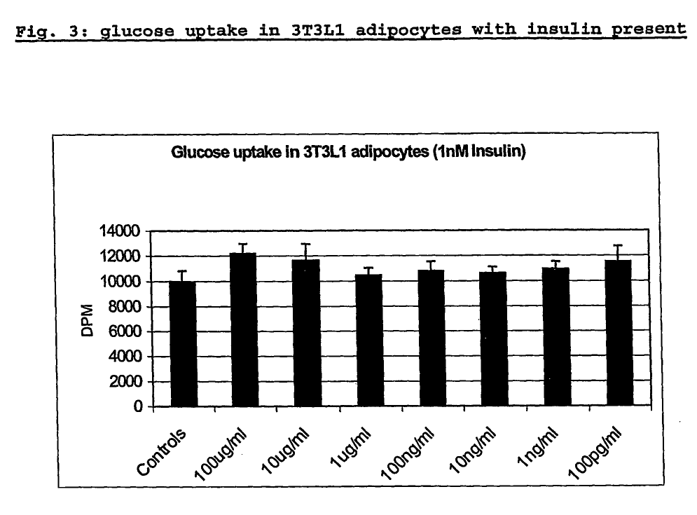

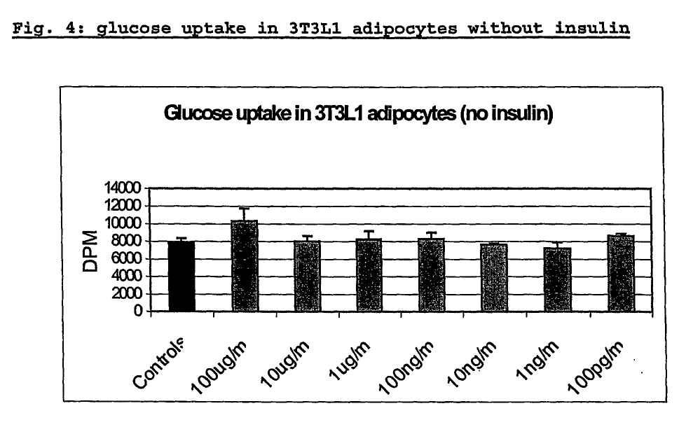

The results represent the mean values of ³H-DPM (decays per minute) and the sample standard deviations for each of the treatments applied in this experiment. Each treatment was tested in triplicate. The results are given in FIGS. 3 and 4 and are shown graphically in Table 1. FIG. 3 shows the effect of the whey protein hydrolysate on glucose uptake in 3T3L1 adipocytes with insulin present and FIG. 4 shows the effect on glucose uptake in 3T3L1 adipocytes without insulin present.

The results show that the claimed whey protein hydrolysates do improve the uptake of glucose in fully differentiated 3T3L1 adipocytes. With the 100 and 10 μg/ml treatments applied in AM supplemented 1 nM insulin, the results indicate 22.27% and 16.70% increase in glucose uptake compared to the experimental controls.

With similar treatments applied in AM without insulin only the 100 μg/ml concentration of Biozate™ 1 indicates increased glucose uptake (31.56%) compared to its experimental control. The above demonstrates that the incubation with the WPH enhances the ability of T₃T adipocytes to take up (3H) glucose. Moreover, in the presence of insulin, the WPH is more effective in stimulating glucose uptake, suggesting that the WPH enhances glucose uptake by sensitising the cells for insulin.

Food Composition Examples

Examples 4 to 6 are of different food compositions that may be used according to the invention.

### Example 4

### Meal Replacement Bar Product

A meal replacement bar product comprising WPH may be prepared according to the formulation below.

| Ingredient | Percentage by weight |
|---|---|
| Honey | 16.0 |
| Sucrose | 10.0 |
| Biozate ™ 1 (WPH) | 13.0 |
| Whey protein | 13.0 |
| Chopped dried fruit and nuts | 10.0 |
| Soy flour | 5.0 |
| Peanut butter | 5.0 |
| Maltodextrin | 4.0 |
| Oats | 6.0 |
| Bran fibre | 2.0 |
| Flavourings | 2.0 |
| Vitamin/mineral premix | 2.0 |
| Chocolate flavoured coating | to 100% wt |

The bar is made by thoroughly mixing together the honey and corn syrup with the peanut butter. The remaining ingredients except the chocolate flavoured coating are added and the mixture is further mixed and formed into a bar shape. To coat it the bar is passed through a curtain of molten chocolate flavoured coating or may be dipped in such a molten coating. The bar is allowed to cool to solidify the coating.

### Example 5

### Ready to Drink Liquid Meal Replacement Product

A meal replacement ready to drink liquid comprising WPH may be prepared according to the formulation below.

| Ingredient | Percentage by weight |
|---|---|
| Water | 75.5 |
| Sucrose | 2.0 |
| Biozate ™ 1 (WPH) | 5.0 |
| Skimmed milk solids | 2.0 |
| High fructose corn syrup | 8.0 |
| Carageenan gum | 1.0 |
| Vegetable oil | 2.0 |
| Caramel flavouring | 1.5 |
| Colourings, other | 1.0 |
| flavourings | |
| Vitamin/mineral premix | 2.0 |

The ingredients were added to the water and the composition was mixed until an homogenous product was obtained.

### Example 6

### Ice Tea Product

An ice tea product comprising WPH may be prepared according to the formulation below. The tea may be made by mixing the ingredients together, with stirring, until a substantially homogenous product is obtained. The product may be cooled as desired.

| Ingredient | Percentage by weight |
|---|---|
| Maltodextrin | 39.4 |
| Tea powder | 9.0 |
| Aspartame | 2.5 |
| Peach flavour | 3.6 |
| N&A apricot flavour | 1.2 |
| Citric acid | 9.0 |
| Magnesium oxide | 0.2 |
| Biozate ™ 1 | 10.0 |
| Vitamin premix | 0.3 |
| Calcium lactate | 23.2 |
| Water | to 100% wt |

1. The use of a whey protein hydrolysate in an edible composition the whey protein hydrolysate being able to induce the cellular release of glucagon-like-peptides and cholecystokinins and/or increasing glucose uptake in target tissues, wherein the whey protein hydrolysate regulates blood glucose levels or results in, or is used for, improving or preventing decline in mental performance and/or for providing a sustained feeling of energy and/or for maintaining or providing a feeling of well-being during the post-prandial period in a subject consuming the composition.

2. The use according to claim 1, wherein the whey protein hydrolysate comprises hydrolysates of β-lactoglobulin, α-lactalbumin or a mixture thereof.

3. The use according to claim 1, wherein the whey protein hydrolysate has a degree of hydrolysis in the range of from 1 to 20%.

4. The use according to claim 1, wherein the whey protein hydrolysate is used in a total amount of from 0.1% to 80% by weight based on the weight of the composition, preferably from 1 to 30% by weight.

5. The use according to claim 1, wherein the edible composition is a meal replacement product or a product to be used as part of a meal replacement diet plan.

6. The use according to claim 5, wherein the meal replacement product or product to be used as part of a meal replacement diet plan is a ready to drink liquid, a liquid produced from a soluble powdered product, a soup, a dessert, a bar, a cereal based or pasta based or noodle based product, or, a soluble powdered product.

7. The use according to claim 1, wherein the edible composition is used as part of a dietary plan or a weight management programme.

8. A method of regulating blood glucose levels, improving or preventing decline in mental performance, providing a sustained feeling of energy or maintaining or providing a feeling of well-being during the post-prandial period, which method comprises the step of orally administering to a subject by means of an edible composition an effective amount of a whey protein hydrolysate which is capable of inducing the cellular release of glucagon-like-peptides and cholecystokinins and/or increasing glucose uptake in target tissues.

9. The method according to claim 8, wherein the whey protein hydrolysate is administered by means of an edible composition.

10. The method according to claim 8, wherein the edible composition comprises a total amount of from 0.1% to 80% by weight based on the weight of the composition of the whey protein hydrolysate, preferably from 1 to 30% by weight.

11. The method according to claim 8 wherein the whey protein hydrolysate comprises hydrolysates of β-lactoglobulin, α-lactalbumin or a mixture thereof.

12. The use or method according to claim 1, wherein the edible composition is selected from dairy based products, soy based products, breads and cereal based products, cakes, biscuits, spreads, oil-in-water emulsions, ice creams, desserts, soups, powdered soup concentrates, sauces, powdered sauce concentrates, beverages, sport drinks, health bars, fruit juices, confectionery, snack foods, ready-to-eat meal products, pre-packed meal products or dried meal products.

13. The use or method according to claim 12, wherein the composition is a meal replacement product or a product to be used as part of a meal replacement diet plan.
